(19) United States
(12) Patent Application Publication
(10) Pub. No.: US 2005/0176053 A1
(43) Pub. Date: Aug. 11, 2005

(54) Method of in situ diagnosis by spectroscopic analysis of biological stain compositions

(75) Inventors: Mark Kent Malmros, Washington Crossing, PA (US); William S. Collins II, San Diego, CA (US); Pier J. Cipriani, Newtown, PA (US)

Correspondence Address: WILMER CUTLER PICKERING HALE AND DORR LLP, THE WILLARD OFFICE BUILDING, 1455 PENNSYLVANIA AVE, NW, WASHINGTON, DC, 20004, US

(73) Assignee: Azurtec, Inc., Washington Crossing, PA (US)

(21) Appl. No.: 11/073,845

(22) Filed: Mar. 8, 2005

Related U.S. Application Data
(63) Continuation of application No. 09/306,662, filed on May 5, 1999, now abandoned.

(51) Int. Cl. C12Q 1/68; G06F 19/00; G01N 33/48; G01N 33/50

(52) U.S. Cl. 435/6; 435/40.5; 702/19

(57) ABSTRACT

A method is disclosed where one or more biological stains are applied to living tissue displaying, or suspected of having cells, or parts of the tissue which are diseased, metaplasic, or otherwise abnormal, including but not limited to lesions which may be thought pre-cancerous or cancerous. The stained tissue is then analyzed, in situ, by reflectance spectroscopy, the results of which are then compared to a digital library of reflectance spectrums of such tissue that have been previously diagnosed by conventional histochemical techniques. The method is further disclosed as a means to monitor the progress of photodynamic therapy using the same biological stains as specific photosensitizers.

### BACKGROUND OF THE INVENTION

Histological examinations play an essential role in therapy. A reliable histopathological diagnosis is an indispensable precondition of the successful treatment of certain diseases and disorders, of which the early recognition of cancerous tissues is of primary importance. Histochemical examination methods should give reliable results easy to evaluate. Moreover, the examination methods should be quick, simple and easy to perform, without requiring specific facilities and training.

The histochemical staining methods known so far do not fulfil these requirements in all respects. The majority of the known staining methods is difficult to perform, time consuming and requires specific attention, and the information supplied is frequently ambiguous. As an example, haematoxylin-eosin test, the most widespread routine method for tissue staining, requires about 90-120 minutes over the freezing and fixing of the section, and the resulting histochemical picture can be evaluated unambiguously only in the case of striking cytological disorders. Otherwise even an approximate diagnosis, involving the risk of serious errors, can be given only after a professional training of several years, and one should frequently rely on standard reference preparations.

Now it has been found and disclosed here that specific biological stain compositions used according to the invention enable one to perform in situ histochemical examinations more quickly, simply and safely, and the resulting histochemical picture enables one to set up a much more reliable diagnosis. Using specific biological stain compositions according to the method invention, histochemical change that could not be detected so far by direct staining techniques, or could not be detected at all can be recognized easily and safely. The invention relates to biological stain compositions for histochemical examinations, in situ, which are then evaluated by means of computer aided reflectance spectrometry. The resulting spectra is then compared by software means to previously diagnosed samples confirmed by routine histochemical methods.

Biological stains have generally been thought to be immutable as to their color, serving as selective visual aids in observing cytological features and not as dynamic chemical entities. Typically, when used as vital stains, they either did or did not stain specific features and mutability of color was not considered nor expected to be a significant feature of the process. If spectral analysis has been used, for example with microscopic spectroscopy, it was to quantify the amount of stain present, particularly in multi stain histochemical processes, and not to make an analytical determination about the underlying cytochemistry. In the present invention the mutability, or change in the color of a metachromatic biological stain or combination of stains, arising from specific cytochemical reactions is evaluated diagnostically. It is further disclosed that the reaction of biological stained tissue and cells to light, or photochromatic response, is dependent on the cytochemical interaction with these stains and is also of diagnostic use.

Furthermore, technology has now become available that make such an approach practical. High performance compact photosensitive arrays combined with economic digital data processing technology to support these arrays and the analysis of the resulting data have only recently become available. Practical application of the instant invention would have been precluded without the significant advances in these areas.

Within the field of histochemistry, and biological stains in general, there is known a class of dyes that are “metachromatic”. From {De Robertis, et al. Cell Biology, 1970, W.B. Saunders Company; pg. 109}: “Some basic dyes of the thiazine group, particularly thionine, azure A, and toluidine blue, stain certain cell components a different color than the original color of the dye. This property, called metachromasia, has interesting histomchemical and physicochemical implications. The reaction occurs in mucopolysaccharides and, to a lesser extent, in nucleic acids and some acid lipids. This reaction is strong in cells that contain sulfate groups (such as chondroitin sulfate), e.g. cartilage and connective tissue.

In mucus-secreting cells, basophilic leukocytes and mast cells, the mucoproteins are not stained the normal color of the dye, but acquire a red violet tint (metachromatic reactions). Some of the intercellular substances that take a similar stain are the matrix of cartilage, tendons, cornea, and the gelatinous substance of the umbilical cord.

Some investigators believe that metachromasia depends on the formation of dimeric and polymeric molecular aggregates of the dye on these high molecular weight compounds. The same basic dyes do not form polymers when acting upon nucleic acid. In this case each cation of the dye combines with one acidic side-chain of the nucleic acid to form a stoichiometrically well-defined salt like compound. A distance of about 5 Å between the anionic groups appears to be necessary for metachromatic staining.”

By way of example, a representative thiazine dye, toluidine blue O (also known as tolonium chloride) has been used as a vital stain for the preliminary diagnosis of pre-cancerous and cancerous lesions in situ; for example in staining oral squamous cell carcinomas. It has also been used in the diagnosis of cervical carcinoma in situ as well as dysplasia and both squamous and basal cell carcinomas of the skin. Its relative specificity for cancerous and precancerous cells arises from the dye's basic affinity for staining nucleic acids, both RNA and DNA. Cancerous and precancerous cells have a very high level of active RNA and DNA metabolism compared to healthy normal tissue and as a result are preferentially stained with toluidine blue O.

Toluidine blue O will rapidly and intensely stain metaplasic, precancerous, and cancerous lesions when applied topically and these lesions are readily seen by the deep blue color. Normal healthy tissue is stained very little if at all. This slight excess stain is easily removed where stained lesions remain (stained) for a period of time. The staining of cancerous and precancerous lesions on application of toluidine blue O occurs within a minute.

There are three mechanisms (not mutually exclusive) of staining that have been suggested by the research to date: 1) mucopolysaccaride staining with metachromasia (a concomitant shift in the absorption spectra of the phenothiazine compound), 2) enhanced nuclei and nuclocoli staining (RNA and DNA rich) associated with enhanced proliferation of these organelles in pre-cancerous and cancerous cells and, 3) enhanced staining of the mitochondria of metaplasic (dysplasic, pre-cancerous, and cancerous) cells.

Canto, et al. (Gastrointest Endosc 1996 July; 44(1):1-7) used methylene blue, another thiazine dye, to selectively stain intestinal metaplasia in Barrett's esophagus with an overall accuracy of detecting specialized columnar epithelium of 95 percent.

In 129 patients with bladder cancer, Fukui, et al. (J Urol 1983 August;130(2):252-5) used 0.2 percent methylene blue as an in vivo staining test. By visual inspection of staining, they were able to pick up 74 and 96 percent of grade 2 and grade 3 tumors respectively. They further assert that the intensity of the stain was correlated with the histologic anaplasia (grade). The blue stain easily identified very small tumors. Using this diagnostic staining method for bladder cancer by extension, in a study of various phenothiazine dyes as photosensitizers for PDT of bladder carcinoma cells by Fowler, et al. (Photochem Photobiol 1990 September;52(3):489-94), it was found that methylene blue was most phototoxic (in vitro) over the use of Azure C, Methylene violet, Thionin, methylene green, and more effective than haematoporphyrin.

Despite the apparent sensitivity of methylene blue for metaplasic cells and tissue, toluidine blue O, also a thiazine dye, has found more widespread application. Sugerman, et al. (Arch Surg 1970 March;100(3):240-3) uses toluidine blue in the diagnostic stain of neoplastic lesions. Chesser, et al. (J Dermatol Surg Oncol 1992 March;18(3):175-6) recommend using toluidine blue 0, ex vivo, as a staining technique for the treatment of adenoid cystic carcinoma by Mohs micrographic surgery.

A specificity of 88 percent and a sensitivity of 92 percent were achieved using a 1 percent solution of toluidine blue in differentiating vulvar intraepithelial neoplasia from non neoplasia epithelial disorders. This in vivo study was conducted by Joura, et al. (J Reprod. Med 1998 August;43(8):671-4).

The use of toluidine blue O in the diagnosis of early stage oral carcinoma is well established in the literature. Warnakulasuriya and Johnson (J Oral Pathol Med 1996 March;25(3):97-103) found a sensitivity of 100 percent for oral carcinomas; for dysplasias, there was found a 20.5 percent false negative rate. The specificity of the technique was only 62% but the over all criteria used in these determinations is unclear. Applying a similar methodology for toluidine blue O in the oral test, Martin, et al. (Oral Surg Oral Med Oral Pathol Oral Radiol Endod 1998 April;85(4):444-6), observed a false negative rate of 42 and 58 percent for carcinoma-in-situ and moderate dysplasia and suggest restricting the use of the method on this basis. It is unclear why detection of these conditions are viewed as a negative result unless the sole objective is detection of a full stage squamous carcinoma.

One aspect of the phenothiazine dye series that has heretofore been overlooked in these vital, or in vivo, staining studies, is the intrinsic property of metachromasia. This property has been studied in the literature:

Thethi, et al. (J Biochem Biophys Methods 1997 Mar. 27;34(2):137-45), demonstrated the use of toluidinc blue's metachromatic shift in measuring cell surface charge. Here, photometric measurements of the metachromatic shift of maximum absorption was used.

The binding of Azure B, another thiazine dye structurally similar to toluidine blue, to chondroitin sulfate was used to investigate this dye's metachromatic structure. Huglin, et al. (Histochemistry 1986;86(1):71-82) were able to distinguish three dye species with absorption bands at 646 nm, 597 nm, and 555 nm.

Using microspectrophotometric measurements, Stockert, et al. (Histochemistry 1991;95(3):289-95), revealed a hypsochromatic shift (from 595 to 570 nm) with toluidine blue staining of isolated metaphase chromosomes.

Scheuner, et al. (Prog Histochem Cytochem 1975;7(2): 1-73), have further studied toluidine blue metachromasia in histological structures. Their analysis suggests that the conditions of attachment of protons to the hydrogen-bearing nitrogen of the dye are largely responsible.

Azariah and Prakasam (Acta Histochem 1975;53(2):182-91) suggest in their studies that toluidine blue may have two sites that are responsible for the exhibition of green and red metachromatic response and that generally salts produce a gamma-metachromatic response.

In developing a spectrophotometric assay for sulfated glycosaminoglycans using 1,9-dimethylimethlylene blue (DMMB) Templeton (Connect Tissue Res 1988;17(1):23-32) found that it exhibited classical metachromasia but suggested a simpler explanation than generally accepted for other thiazine dyes such as Azure A. This dye dimer (DMMB) reacts with the polyanion to produce a single metachromatic species by ionic perturbation of the chromophore. Dimer disruption, such as with nucleotides, induce metachromasia in the absence of polyanion.

Stain compositions prepared by the oxidation of methylene blue was studied by thin layer chromatography. Marshall (Histochem J 1976 July;8(4):43142) found that various named methods for the production of Polychrome methylene blue, Azure A, Azure B, Azure C and methylene violet give complex mixtures of up to eleven dyes. Ten of them can be identified by their visible absorption spectra including sym-dimethylthinonine, thinonin and thinonoline and thionol.

From a selection of cationic dyes, viz. Thionin, Safranin O, toluidine blue O, dimethylmethylene blue, Cuprolinic blue and others, Kiraly, et al. (Histochem J 1996 August;28(8):577-90 used microspectrophotomerty for semiquantitative estimation of glycosaminoglycans in histological sections of articular cartilage. They found that Thionin and Safranin O gave the best results as cationic dyes due to the linearity of the staining intensity curves.

In vivo reflectance photometry has found limited use. However, Troilius and Ljunggreen (Br J Dermatol 1995 February;132(2):245-50) measured the therapeutic progression of port-wine stain (PWS) during the course of laser therapy on sixty-six patients. This simply evaluated blanching of the pigmentation of the skin associated with PWS syndrome.

One aspect of the present invention utilizes this inherent property of metachromasia found in the phenothiazine dyes by example to enhance the diagnostic utility and specificity in the detection of metaplasia by spectrometrically analyzing stain metachromasia in situ.

The histochemical pathology of dysplasic, pre-cancerous, and cancerous lesions that would expected to be stained with any of the thiazine dyes, will vary as to the degree of metachromasia within the cell tissue layer. This depends on the variation in mucin production, aberration of nuclei, nucleoli, and mitochondrial organelle distribution, as well as changes in cell membrane permeability, charge structure and membrane transport properties, etc. with the various cells types associated with each diagnosis and the stage of metaplasia or cell transformation.

Zhou, et al. (Med Phys 1996 December;23(12):1977-86) present a technique for multiple wavelength image acquisition and spectral decomposition based upon the Lambert-Beer absorption law. This algorithm is implemented based on the different spectral properties of the various stain components. By using images captured at N wavelengths, N components with different colors can be separated. This algorithm is applied to microscopy images of doubly and triply labeled prostate tissue sections.

The (reflectance) spectroscopic analysis of lesions that stain with toluidine blue or with other biological stains or dyes, or with a combination of such stains or dyes (which may or may not necessarily include a metachromatic stain) allow for a differential diagnosis of the underlying disease, or disease state of the stained lesion. Cells displaying various stages of metaplasia stain differentially, from a combination of biological stains, which is then correlated to the spectrum with a high degree of specificity. This is accomplished by comparing the reflectance spectrum of the stained tissue or lesion with a “library” or composite of spectrums from lesions that have been similarly stained and subsequently diagnosed by conventional or classical histochemical methods. This is best accomplished by the use of state of the art spectrometers and microprocessor based computers that acquire the spectroscopic data and compare it graphically and/or otherwise by software techniques to the digital spectrum library of “stained lesions and tissues”. This also includes, for example, a software means of subtracting (from the stained lesion spectra) the background reflectance spectra of normal tissue both with and without stain, including a baseline spectrum of the patient's normal tissue. A simple spectrometer may be comprised of a diffraction grating and a linear CCD (charge coupled device) array, which intercepts the dispersion from the grating, through which the reflected light passes; being brought from the illuminated and stained lesion to the spectrograph by means of an optic fiber or bundle of optic fibers.

U.S. Pat. No. 5,832,931 teaches a method for the detection of molecular diagnostic agents by means of photoactivation. Specific light signals are detected in this method from either endogenous photosensitive chemicals or specific entities that are introduced to produce a desired “two-photon” active agent. On specific photo-activation, the molecular entity is then detected by emitting energy characteristic of the activated molecular entity. However, this method does not utilize analysis in evaluating the underlying cytochemical or histochemical changes that would correspond to the stage of metaplasia by means of characterizing the spectrum of the photo-active molecular species; or as a means of analysis to differentiate between tissue or cells undergoing normal repair processes and metaplasic cells.

U.S. Pat. No. 5,697,373 teaches the use of fluorescence or Raman spectroscopy, or combination thereof as a mean of diagnosing cervical precancers. The fluorescence relies on the inherent chemistry of the tissue or cell. Further, it neither relies on endogenous molecular entities or analysis of spectra underlying or correlating with a metaplasic stage as the diagnostic means.

U.S. Pat. No. 5,131,398, for both a method and an apparatus, again uses endogenous fluorescence excited at one wavelength and detected at two different wavelengths to distinguish between benign or normal tissue and cancerous tissue.

The analysis of tissue and cells in vivo by complex means are taught in U.S. Pat. No. 5,800,350. An apparatus is described providing for a plurality of different stimuli such as electrical, light, heat, sound, magnetic and to subsequently detect plural physical response to these stimuli. Software means of analyzing the data are used to categorize the responses. However, specific biochemical stains are not employed or considered in this apparatus.

U.S. Pat. No. 5,748,162 teaches of multi variant spectral bio-imaging analysis for diagnostics and therapy utilizing optical means including two-dimensional photodetector arrays. Requiring sample preparation and visualization, the method incorporates in part fluorescent dyes to enhance imaging but makes no reference to the utility of spectrum analysis of metachromasia or differential biological staining of tissue or cells as a means of correlating metaplasic stage.

U.S. Pat. No. 4,973,848 uses a pair of laser beams in the course of photodynamic therapy where one beam is used to analyze the surface to be treated as a means of controlling the properties of the “treatment” beam but makes no reference to the utility of spectrum analysis of metachromasia or differential biological staining of tissue or cells as a means of correlating metaplasic stage. Further the analysis is limited to the wavelength of the analysis laser beam rather than a broad spectrum of light energy.

Complex biological stain compositions for histological examinations are taught in U.S. Pat. No. 4,595,582. These dyestuff compositions, of which some components are of the thiazine family, are an improvement to conventional histochemical methods, enhancing visualization of cytological structure within fixed tissue. It does not teach or make reference to the utility of spectrum analysis of metachromasia or differential biological staining of tissue or cells as a means of correlating metaplasic stage.

The advantage of the instant method of in situ diagnosis of diseased tissue is the reduction or elimination of the process of surgically removing tissue and applying conventional histochemical methods before a diagnosis can be rendered. It is thus a faster method and to an extent safer than the surgical removal of tissue. It provides for “at the point” means of making a diagnostic decision where it might otherwise be inconvenient or impossible to make such determinations for reasons of costs, time, or availability of facilities.

The above-described disclosure also finds utility in application to “photodynamic therapy”.

Photodynamic therapy (PDT) is a non-surgical procedure that uses a chemical or biochemical photosensitizer to target cancerous and precancerous cells which are subsequently irradiated with a high intensity light source or laser to activate the photosensitizer and kill the target cancer or precancerous cells.

The utility of combining in situ reflectance spectrometry with photodynamic therapy has been indirectly suggested here above in the brief review of the literature; specifically the use of methylene blue for diagnosis of bladder cancer (Fukui, et al.; J Urol 1983 August;130(2):252-5) and in PDT of bladder cancer by Fowler, et al. (Photochem Photobiol 1990 September;52(3):489-94).

Photodynamic therapy is an ancient concept and has been described and utilized over 30 centuries ago. The therapy was used in ancient times for the treatment of vitiligo in India, China and Egypt. In the last century, ultraviolet (UV) radiation was successfully used in the treatment of lupus vulgaris, a type of skin tuberculosis endemic in the Scandinavian countries. PDT usually involves the administration of one or more photoactive agents to the subject to be treated followed by exposing the specific target location or target organ of the subject to light.

Thus, for example, upon illumination, Methylene Blue has been used to kill Trichoderma Viride, a common fungus, outside the body. Similarly, acridine orange, as well as Methylene Blue, kills blood fluke Schistosoma mansoni, in vitro, upon exposure to light. [P. S. Lacaz and J. C. E. Holanda, Bol. Acad. Nac. Med. (Brazil) 145:43 (1974), Chem Abstr. 86:134166.) Likewise, larvae of Anopheles mosquitoes are killed by the simultaneous exposure to photoactive dyes and light. [A Barbieri, Accion fotodynamica de la luz. Riv. Malariol, 7:456 (1928);] [H. Schildmacher, Biol. Zentr. 69:468 (1950).] PDT has been used to neutralize externally the toxicity of many snake venom without significantly altering their antigenicity so that they can still be used to manufacture antibodies for the snake venom [W. F. Kocholaty, J. C. Goetz, et al., Toxicon 5:153 (1968).] Similar results have been reported for some animal viruses. Thus, utilizing similar technique, vaccines, including influenza vaccines, have been prepared. [J. D. Spikes and R. Livingston, Adv. Radiat. Biol. 3:29 (1969);] [C. V. Hanson, in “Medical Virology,” Proc. Int'l Symp. 2:45, Elsevier (1983).] Likewise, influenza or encephalomyelitis viruses externally added to contaminate human blood plasma are inactivated by light in the presence of toluidine blue dye without significant alteration to the properties of plasma proteins. [F. Heinmets, J. R. Kingston and C. W. Hiatt, Walter Reed Army Institute of Research Report 53-55:1 (1955).]

Extracorporeal PDT, utilizing light and psoralen dyes, has also been reported for the treatment of cutaneous T-cell lymphoma. Psoralen dyes in the presence of light have also been used for the treatment of vitiligo. [T. B. Fitzpatrick and M. A. Pathak, J. Invest. Dermatil. 32:229 (1959);] [A. V. Benedetto, Cutis 20:469 (1977).] Skin tumors have been treated with the simultaneous exposure of the tumors to both eosin dyes and light. [H. V. Tappeinci and A. Jesionek, Munch. Me. Wochenschr. 50:2042 (1903).] In the early 40's, it was observed that hematoporphyrin derivative (hereinafter Hpd) preferentially accumulated in tumors and lymph nodes. [H. Auler and G. Banzer, Z. Krebforsch. 53:65 (1942).]

As a result, methods have been developed to capitalize on the unique property of Hpd as a tumor marker in the detection and localization of different forms of cancer cells. [E. G. King, et al., Hematoporphyrin Derivative as a Tumor Marker in the Detection and Localization of Pulmonary Malignancy, in Recent Results in Cancer Research. Vol. 82, Springer-Verlag, Berlin-Heidelberg, 1982, 90;] [R. D. Benson, et al., Mayo Clinic Proc. 57:548 (1982).] Although the unique photodynamic properties of Hpd, as well as its unique preferentlial affinity toward tumor cells, had long been known, it was more than half a century later that the potential of using Hpd to selectively destroy tumor cells was explored. In 1966, Lipson and co-workers reported treating one case of recurrent breast cancer using a combination of Hpd and light. [M. S. Lipson, M. J. Gray and E. J. Baldes, Proc. 9th Intl. Cancer Congr., p. 393 (1966).] The use of light in the presence of Hpd to selectively destroy tumor cells in human has been reviewed by Dougherty et. al. [T. J. Dougherty, et al., Photoradiation Therapy: Clinical and Drug Advances. In Porphyrin Photosensitization, D. Kessel and T. J. Dougherty, Eds. Plenum Press, N.Y., pp. 3-13, 1983.]

U.S. Pat. No. 4,649,151 teaches the preparation and purification of porphyrin-type drugs. The patent also teaches the diagnosis and destruction of cancer cells with porphyrin-type drugs. In treating humans or other mammals with the drugs, light must be irradiated on the cancer cells in such a position as to uniformly illuminate the cancer cells. When cancer cells, having the porphyrin-type drugs associated therewith, are illuminated with light, the drugs are activated and thus causing the destruction of the cancer cells by a mechanism not completely understood yet. The patent also discloses several apparatus for transmitting light to different parts of the body.

U.S. Pat. No. 4,614,190 discloses that while a dye such as Hpd is being held within the tumor cells in the body, the activation of the dye is accomplished by pulsed electromagnetic radiation.

U.S. Pat. No. 4,727,027 teaches the inactivation of pathogenic biological microorganisms by simultaneous treatment with furocoumarins and a long wavelength ultraviolet light to under conditions that limit the availability of oxygen and other reactive species.

Cyanine dyes are members of another class of dyes that are selectively retained by tumor cells and certain viruses. For example, Merocyanine 540, (commonly referred to as MC 540) has been used for light-induced tumor and viral chemotherapy. [K. S. Gulliya, J. L. Matthews, J. W. Fay, and R M. Dowben, Proc. SPIE-Intl. Soc. Opt. Engineering 84f7:163-65 (1987);] [K. S. Gulliya, S. Pervaiz, D. G. Nealon, and D. V. VanderMeulen, Proc. SPIE-Intl. Soc. Opt. Engineering 907:34-36 (1988);] [F. Sieber, Photochem. and Photobiol. 46:103542 (1987).]

The emphasis on using a photoactive compound or dye as the photoactivating or light-activating compound in photoradiation of tumors or viruses, bacteria, and fungi (hereafter collectively “microorganisms”) is based on two important properties of the photoactive compound or dye. Firstly, the photoactive compound or dye is preferentially accumulated and retained to a higher degree in or around the target tumor or microorganisms than in the surrounding normal body tissues. Secondly, after being retained in or around the tumor or virus, the photoactive compound or dye is properly photoactivated causing the destruction of tumor cells or microorganisms with which the dye has associated.

The destruction of tumor cells or microorganisms occurs when they are simultaneously exposed to the dye and light of a suitable wavelength. The generally accepted mechanism of cell kill by photoactivated dye is that when activated by appropriate light, the dye undergoes an energy transfer process with oxygen to form a reactive singlet oxygen, which subsequently oxidizes and kills the cell or microorganism to which the dye has attached or associated as a substrate. [K. R. Weishaupt, C. J. Gomer, and T. J. Dougherty, Cancer Res. 36:2326-29 (1976);] [F. Sieber, Photochem. and Photobiol. 46:1035-42 (1987).]

The life-time of the extremely reactive singlet oxygen is extremely short, less than a fraction of microsecond. Hence, the currently accepted method of practicing PDT is to first let the photoactive compound bind to the target tumor cells or microorganisms and then activate the bound photoactive compound. Thus, when the reactive singlet oxygen is generated from photoactivation, the target tumor cells or viruses that are in the close proximity to the activated dye and oxygen are destroyed. The normal cells do not preferentially accumulate the photoactive compound, hence generally very little reactive singlet oxygen is generated in their close proximity. Accordingly, the normal cells are generally spared from destruction by the photoactivated photoactive compound. T. J. [Dougherty, et at., Photoradiation Therapy: Clinical and Drug Advances. In Porphyrin Photosensitization, D. Kessel and T. J. Dougherty, Eds. Plenum Press, N.Y., pp. 3-13, 1983.]

In the application of photodynamic therapy, these photo sensitizers may be analyzed during the procedure by utilizing reflectance spectrometry to follow the course of the therapy during the photo irradiation. This would provide for an analytical means of determining the efficacy and end-point of the procedure assuring complete a method as might otherwise be possible.

The phenothiazine family of dyes has found use as photosensitizers in PDT. For example. Konig et al. (J Cancer Res Clin Oncol 1987;113(3):301-3), evaluated the photodynamic cytotoxic effect of Methylene blue using mice with solid Ehrlich carcinomas. Using laser radiation emitting at 647 nm 676 nm, they achieved a significant tumor destruction, including complete tumor destruction. They emphasize the importance of using the red spectral range as being more readily transmitted through tissue.

Kleemann (Laryngorhinootologie 1990 August;69(8):437-9) confirms this result using Methylene blue as a photosensitizer for photodynamic therapy of malignant tumors of the mouth cavity, larynx, and pharynx. The combination of both red laser light and the photosensitizer is emphasized.

Darzynkiewicz, et al. (Cancer Res 1988 Mar. 1;48(5):1295-9) evaluated the photosensitizing effects of the tricyclic heteroaromatic cationic dyes pyronin Y and toluidine blue O (tolonium chloride). Using Pyronin Y [3,6-bis(dimethylamino) xanthylium chloride; PY] and toluidine blue O [tolonium chloride; 3-amino-7-(dimethylamino)-2-methyl phenotliiazin-5-ium chloride; TB], cationic dyes commonly used in cytochemistry that have affinity to nucleic acids, predominantly to RNA. In live cells these dyes accumulate in mitochondria and sensitize the cells to light. The photosensitizing effects of PY and TB were compared with those of another if mitochondrial cationic dye, rhodamine 123, and a noncationic dye, merocyanine 540, which binds to the cell membrane. Ninety % reduction of clonogenicity of human epidermoid carcinoma (A-253) cells was achieved by cell exposure to 0.7, 1.0, 1.2, or 1.5 J/cm2 doses of white light, respectively. The data suggest that PY and TB, like other mitochondrial dyes, may have a selective antitumor photosensitizing activity. The dyes were used singly and were not used in any diagnostic modality. Pyronin Y has a peak absorption at 552 nm whereas toluidine blue O has a peak absorption at 640 nm.

Orth et al. (Chirurg 1995 December;66(12):1254-7) used methylene blue in the photodynamic therapy of small adenocarcinomas. The animal experiments showed a tumor volume reduction of 1:12, as compared to a control group, two weeks after the first PDT-application. After the second PDT-treatment 6 out of 10 tumors were destroyed. Four carcinomas showed inhibited growth after the treatment. The method was clinically applied in 3 patients with local tumor recurrence in the area of the anastomosis after esophagus resection. 72 hours after PDT-treatment 4-5 mm tumor necrosis could be proven experimentally. PDT was repeated at the same site within 2 weeks. There were no experimental or clinical complications during or after PDT. The treated tumor areas showed no local tumor growth within 6 months after PDT-treatment.

Canete et al. (Anticancer Drug Des 1993 December;8(6):471-7), made a comparative study of the uptake and photoinactivation of HeLa cells treated with methylene blue (MB) and toluidine blue (TB). Cell toxicity induced by different concentrations of either MB or TB showed that 10(−5) M was the concentration at which dark damage was not observed, while an elevated photoinactivation could be detected with both thiazines. The uptake studies showed that the penetration kinetics of 10(−5) M MB into HeLa cells is faster than that of TB, used at the same concentration, reaching saturation after 6 or 12 h of incubation, respectively. For both sensitizers, the survival of HeLa cells was dependent on the incubation time, as well as the light dose, for a given concentration. They suggest that cell photoinactivation produced by MB was higher than that produced by TB. Wainwright, et al. (FEMS Immunol Med Microbiol 1997 September; 19(1):75-80) studied the photobactericidal activity in a closely related series of commercially available phenothiazinium dyes The photosensitisers were illuminated using a non-laser light source at a fluence of 1.75 mW cm-2 and this resulted in the enhancement of antibacterial activity in liquid culture.

Wainwright. Et al. (FEMS Microbiol Lett 1998 Mar. 15;160(2):177-81); the photobactericidal activity of phenothiazinium dyes against several pathogenic strains of Staphylococcus aureus, four of which were methicillin-resistant resulted in the enhancement of antibacterial activity in liquid culture and in greater efficacy than the methicillin analogue flucloxacillin. For methylene blue, dimethyl methylene blue and new methylene blue illumination led to increases in bactericidal activity <or =16-fold, typically 4-fold. In addition dimethyl methylene blue and new methylene blue were active against epidemic strains of methicillin-resistant Staphylococcus aureus at concentrations lower than that of vancomycin (>or =0.5 microM).

Martin, et al. (Arch Biochem Biophys 1987 July;256(1):39-49), looked at representative thiazines, xanthenes, acridines, and phenazines, which they found photosensitized the oxidation of reduced pyridine nucleotides and reduced glutathione when illuminated with low intensity visible light. Photooxidation resulted in oxygen consumption and in superoxide generation, assayed as the superoxide dismutase (SOD)-inhibitable reduction of ferricytoclirome c.

Stockert, et al. (Cancer Chemother Pharmacol 1996;39(1-2):167-9) analyzed possible alterations of the microtubule cytoskeleton of cultured cells subjected to photodynamic treatments with the thiazine dyes methylene blue or toluidine blue. Untreated control cells showed the normal distribution of interphase microtubules, whereas considerable or severe disorganization of the microtubule network was observed after photodynamic treatments.

Paardekooper, et al. (Photochem Photobiol 1995 January;61(1):84-9) studied the positively charged photosensitizer toluidine blue (TB) and found it can induce loss of clonogenicity in Kluyveromyces marxianus. As a consequence of the localization of this dye at the cell surface, photodynamic action results in extensive damage at the level of the plasma membrane. It is shown that treatment with TB and light resulted in the inhibition of alcohol dehydrogenase, cytochrome c oxidase, glyceraldehyde-3-phosphate dehydrogenase and hexokinase. Photodynamic treatment also lowered the ATP levels. The ATP levels could be partially restored in the presence of glucose but not with ethanol. Toluidine blue binding experiments revealed that photodynamic treatment caused a rapid increase in the amount of cell-associated dye. Moreover, it also appeared that this treatment decreased the binding of TB3 to the cell surface. It is concluded that TB enters the cell during the first minutes of illumination, whereafter intracellular enzymes are inactivated. The data indicate that photodynamic damage of intracellular sites contributes to the loss of viability.

Wilson, et al., (J Oral Pathol Med 1993 September;22(8):354-7) sensitized Candida albicans, and other Candida spp. responsible for HIV-associated candidosis to killing by low-power laser light with thiazine dyes. Suspensions of C. albicans were treated with a number of potential photo sensitisers, exposed to laser light from a Helium/Neon (HeNe) or Gallium aluminum arsenide (GaAs) laser for 120 s and survivors enumerated. Toluidine blue O (TBO), thionin, and crystal violet were able to sensitize the yeast to killing by light from the HeNe laser (energy dose=876 mJ at a density of 66.36 J/cm2), the kills achieved being 6.8×10(6) cfu/ml, 3.1×10(6) cfu/ml and 1.3×10(6) cfu/ml respectively. TBO was also able to sensitize several other Candida spp. to killing by HeNe laser light. Dihaematoporphyrin ester was not an effective photosensitizer under the conditions employed. Methylene blue, but not aluminum disulphonated phthalocyanine, was able to sensitize C. albicans to killing by light from the GaAs laser (energy dose 1.32 J at a density of 2.04 J/cm2). The viability of the yeast was not affected by exposure to laser light in the absence of the photosensitisers.

### BRIEF SUMMARY OF THE INVENTION

The use of biological stains in direct staining in vivo has been shown to have a high degree of sensitivity to a variety of metaplasic, precancerous and cancerous cells and tissues. For example, the thiazine dyes toluidine blue O and methylene blue have found frequent use in the in vivo diagnosis of oral epithelial carcinomas, dermal epithelial carcinomas, esophageal cancer, cervical and vaginal cancers, and even in the detection of bladder cancers. However, the specificity of the staining process in differentiating between the stage and type of metaplasia has been variable and has not allowed for a definitive diagnosis of the Disease State. Generally, vital or in vivo staining has not been able to distinguish between normal cellular repair processes and metaplasia. Practitioners have used the sensitivity of the stains to locate diseased tissue and then subsequently relied on biopsy and classical histochemical techniques for a final diagnosis. Histochemical methods further rely on staining the morphological as well as the biochemical features retained after the fixation and sectioning of the tissue sample. The staining features of intensity and color are then examined and a subjective if skilled determination as to the underlying cell type is rendered as the diagnosis.

In the present invention specific biological stain compositions, comprised of one, or more than one, stain are applied directly to living tissue suspected of some underlying disease state. The properties of the stain compositions including concentration, pH, stain ratio and other solvent characteristics are controlled. Application would include for example preparing the area to be stained by a suitable cleaning procedure such as swabbing the area with an alcohol preparation. In advance of the application of the specific stain composition, the area to be stained is then measured by reflectance spectrometry, one or more times, to determine a background spectrum over the range of wavelengths for which the whole analytical method is optimal.

The reflectance spectrometry can be of conventional design and may, for example, be comprised of a fiber optic bundle through which both an illuminating source of light passes to irradiate the area being analyzed and through which the light reflected is collected and passes to the spectrometry proper. The reflected light is directed through a collimating slit and then to a diffraction grating. The dispersion of light by the diffraction grating is intercepted by a linear array of charge coupled devices (CCD) with suitable sensitivity over the range of wavelengths to be measured. An analog and digital electronic processing device is used to connect to or otherwise interface with a microcomputer supporting the charge-coupled device. The microcomputer renders the data collected from the CCD array by software means into a graphically plot of intensity versus wavelength of light.

Spectrometry technology is not specific to this invention and is readily available from a number of instrumentation manufacturers at the time of this invention. It can be readily seen by those skilled in these arts that the spectrometry can be obtained in a variety of ways that may be anticipated under the present invention. For example, a prism may replace the diffraction grating for the dispersion of the light spectrum. The CCD array may be replaced with an imaging CCD array or may be replaced by a single photocell that can be moved along the dispersed spectrum, and so forth. A single electronic photoreceptor may be used with a combination of filters (in lieu of dispersing the reflected light into a spectrum of light), passing specific wavelengths or bands of wavelengths which may then be compared as a spectrum suitable to the purposes and objectives set forth in this specification.

The stain composition is applied by suitable means such as an applicator or wipe and allowed to remain on the desired area for a specified time. Excess stain is subsequently removed, again with a wipe or other means of de-staining those areas which do not retain the stain by biochemical or cytochemical means. The area stained is then measured by reflectance spectrometry, one or more times, to collect the desired stain spectrum over the range or wavelength, for which the whole analytical method is optimal.

The analysis of the reflectance spectrum of the stained tissue area by software means is significant to the present invention. It may be conducted in a variety of ways to fulfill the intent of this method. For example, the software analysis of the spectra may compare the metachromatic shift of the stain toluidine blue O between two or more specific wavelengths by correlation. The results are then compared to a body of data previously collected and correlated to underlying conventional histochemical data defining the cellular stage of metaplasia. It can readily be seen that a combination of two stains, for example methylene blue and pyronin Y, applied either from one composition or applied as two separate preparations and analyzed by comparative spectrometric means and suitable software analysis may afford a desirable degree of enhanced specificity in certain applications. The two stains would compete for certain histochemical features that would distinguish cells undergoing normal repair processes and those cells that are metaplasic or neoplasic. Further additional diagnostic utility may be afforded by means of monitoring the photo oxidation of the specific stain or stain composition by spectrometric means and analyzing the resulting change in the spectra, correlating the result to underlying clinical analysis of the procedure verified by conventional means. The photo oxidation may be by means of a broad-spectrum high intensity light source (either through the fiber optic bundle or external to the fiber optics), a filtered high intensity light source, or by means of a specific wavelength laser. In this manner of analysis, the change in the characteristic spectrum of the measured tissue stain combination (in other words, the photo bleaching process; i.e. “photochromasia”.) may be followed as a function of time, intensity, or a combination thereof.

For certain stain compositions, the characteristic differential spectrums that are empirically determined by this method in specific applications may allow for the use of a reflectance photometer and one or more wavelength band pass filters to correlate the diagnostic results in lieu of a reflectance spectrometer.

It can be further seen that this in situ diagnostic method may be extended to a highly controlled photo therapeutic method for the destruction and removal of diseased tissue and cells. It has been seen that many biological stains, for example O, has found application as photosensitizers for photodynamic therapy. Because of the specificity of these biological stains for metaplasic cells, including cancerous cells, the photo oxidation results in a cytotoxic effect. It may be highly advantageous to the practitioner to be able to assess the cytological state of an area of tissue in applying irradiation for photo therapeutic means, for example allowing discrimination between highly metaplasic tissue as compared to inflamed but otherwise normal tissue area and cells undergoing the normal repair processes. This would minimize for example the amount of tissue destruction by means of a high power laser. Further, the course of the phototherapy up to a defined end point can be accurately and carefully assessed using the combination of biological stain/photosensitizer and a spectrometer means of analyzing the stain in situ.

1-19. (canceled)

20. A method for diagnosing dysplasia, pre-cancer or cancer in situ in biological tissue or cells of a living organism, comprising:
a) applying to the tissue or cells in situ a dye selected from the group consisting of methylene blue and toluidine blue O;
b) removing excess dye from the tissue or cells;
c) generating a reflected light spectrum from the tissues or cells by illuminating the stained tissue or cells with light;
d) directing the reflected light spectrum to a spectrometer;
e) comparing the degree of the metachromatic shift of the dye from the reflected light spectrum of the stained tissue or cells with the degree of the metachromatic shift of the dye from a library of previously obtained spectra of similarly stained tissue or cells that have been previously diagnosed by conventional methods and exhibit the particular disease state being diagnosed, wherein said tissue or cells in the library are stained according to the same protocol as the stained tissue or cells to be diagnosed; and
f) correlating the degree of the metachromatic shift of the dye from the reflected light spectrum with a disease state, said disease state selected from the group consisting of dysplasia, pre-cancer and cancer, whereby an in situ diagnosis of dysplasia, pre-cancer or cancer is made.

21. A method as in claim 1, wherein said comparing comprises the use of a digital microprocessor.

22. A method as in claim 1, wherein the tissues or cells are thought to be metaplastic.

23. A method as in claim 1, wherein the spectrometer is able to measure light for a range of or some part of a range of wavelength from 200 to 1100 nanometers.

24. A method as in claim 1, wherein the reflected light spectrum is measured and recorded, and said measuring comprises the use of a photometer and one or more light filters.

25. A method as in claim 1, wherein the tissue or cells are from at least one organ selected from the group consisting of skin, cervix, vagina, mouth, colon, and esophagus.

26. A method as in claim 1, wherein, prior to said comparing step a reflected light spectrum from unstained tissue or cells is subtracted from the spectrum of the stained tissue or cells.

27. A method as in claim 1, wherein the tissue or cells are from an internal organ.
